(19) United States
(12) Patent Application Publication
(10) Pub. No.: US 2023/0414844 A1
(43) Pub. Date: Dec. 28, 2023

(54) CROSS-LINKED HYDROGEL MICRONEEDLE FOR TRANSDERMAL DRUG DELIVERY AND METHOD FOR PREPARING THE SAME

(71) Applicant: Hangzhou Singclean Medical Products Co., Ltd, Hangzhou (CN)

(72) Inventors: Zhihong Zhang, Hangzhou, Zhejiang (CN); Qingzhi Deng, Hangzhou, Zhejiang (CN); Yufei Xiong, Hangzhou, Zhejiang (CN); Wei Huang, Hangzhou, Zhejiang (CN)

(21) Appl. No.: 17/899,612

(22) Filed: Aug. 31, 2022

(30) Foreign Application Priority Data
Jun. 24, 2022 (CN) 202210729218.5

(51) Int. Cl. A61L 31/14; A61L 31/04

(52) U.S. Cl. CPC A61L 31/145; A61L 31/042; A61L 31/048

(57) ABSTRACT

The present disclosure discloses a cross-linked hydrogel microneedle for transdermal drug delivery and a method for preparing the same. The preparation method includes the following steps: mixing a polyvinyl alcohol solution and a hyaluronic acid solution; freeze-thawing the mixed solution in a mold to obtain a gel with no fluidity; and demolding the gel and further performing irradiation cross-linking to obtain the cross-linked hydrogel microneedle. The preparation method described in the present disclosure overcomes the problem of poor stability that exists in the preparation by a freeze-thaw method and the problem of easy introduction of air bubbles that exists in the preparation by an irradiation method, and can increase the yield of the microneedles, which is conducive to mass production. The prepared microneedles can be transported and stored for a long time and have wide application prospects.

### CROSS-REFERENCE TO RELATED APPLICATION

The present application claims priority from Chinese Patent Application No. 202210729218.5 filed on Jun. 24, 2022, the contents of which are incorporated herein by reference in their entirety.

### TECHNICAL FIELD

The present disclosure belongs to the technical field of medical cosmetology and relates to a soluble hydrogel microneedle and a method for preparing the same.

### BACKGROUND

In the cosmetic and medical industry, for operations involving subcutaneous implantation, such as minimally invasive surgery, drug delivery and subcutaneous sampling, a common method is needle-based injection or extraction. Needle-based injection will produce sensory pain and cause greater psychological pressure to the patient. In addition, improper use of needle syringes may cause infection or allergies, causing certain safety hazards. Transdermal Drug Delivery System or Trandermal Thrapeutic System (TTS for short) is a method for absorbing drug through the skin. After the drug is absorbed through the skin, it enters the blood circulation of the human body, reaches an effective blood drug concentration, and realizes disease treatment or prevention. Compared with traditional oral drug delivery, it avoids liver first-pass effect, gastrointestinal inactivation and the like; and compared with injection drug delivery, it avoids the pain caused by subcutaneous injection and improves patient compliance. However, the tight stratum corneum in human skin not only plays a protective role, but also hinders the absorption of drugs. As an effective and minimally invasive novel mode of transdermal drug delivery, microneedle technology has attracted more and more attention for its application potential. Microneedles have the advantages such as painless, minimally invasive and efficient drug delivery, which can enhance transdermal drug delivery by piercing human skin, destroying skin barrier (stratum corneum) and improving skin permeability. This technology has been shown to increase transdermal delivery of a variety of biomolecules, including small molecule drugs, vaccines, proteins, DNA and the like.

Hydrogels, three-dimensional polymeric materials with chemical or physical cross-linked structures, have been used as novel microneedle materials. Among them, polyvinyl alcohol, as a polymer material with good biocompatible mechanical strength, can be cross-linked into hydrogel through freeze-thaw, irradiation and other means without adding a cross-linking agent, and thus can be applied to the field of hydrogel microneedle. The hydrogel microneedles formed by polyvinyl alcohol are in a rigid glassy state when it is dried, and can be easily pierced into the skin. After piercing into the skin and absorbing the liquid, they change from the glassy state to a soft gel state, therefore avoiding damage to the subcutaneous cells, eliminating the hidden danger of breaking the rigid microneedles under the skin, causing no significant pain, causing minimal damage to the tissue during use, and posing minimal threat to the environment after treatment.

In recent years, in-depth study on preparation of polyvinyl alcohol microneedle products by the freeze-thaw method has been conducted in China. Among them, Chinese patent with publication number of CN109701152A introduces a soluble microneedle patch prepared from polyvinyl alcohol-dextran solutions with different mass fractions via centrifugation, freezing and thawing, which has good mechanical properties and can pierce the skin; and Chinese patent with publication number of CN110090044A introduces a hydrogel microneedle patch prepared by mixing polyvinyl alcohol and a highly hydrophilic polymer solution, repeatedly freeze-thawing the resulting mixed solution to form cross-linked ice crystals of polyvinyl alcohol, drying and then demoulding. During use, the hydrogel microneedle patch with extracted skin interstitial fluid is immersed in a buffer solution and warmed to a suitable temperature to dissolve the cross-linked ice crystals of the hydrogel microneedle patch in the buffer solution, so as to release the target substance.

However, the cross-linked network structure formed by physical cross-linking through the freeze-thaw method alone is unstable and varies with temperature, and the hydrogel will be thawed when the temperature rises to a certain level. Therefore, the microneedle products prepared by the freeze-thaw method cannot meet the requirements of long-term transportation and storage. When an irradiation method is used for cross-linking, the stability of the prepared hydrogel is improved, but the flowing uncross-linked solution needs to be fixed in a mold for irradiation, and air bubbles are easily brought in during the transport process, which affects the state and properties of the product. In addition, the molds take up a lot of volume, which reduces the yield and increases the difficulty of mass production.

### SUMMARY

The purpose of the present disclosure is to provide a cross-linked hydrogel microneedle for transdermal drug delivery and a method for preparing the same, so as to overcome the problem of poor stability for the preparation by a freeze-thaw method and the problem of easy introduction of air bubbles for the preparation by an irradiation method.

According to a first aspect of the present disclosure, the following technical solutions are used in the present disclosure.

A method for preparing cross-linked hydrogel microneedles for transdermal drug delivery, comprising subjecting a mixed solution of sodium hyaluronate and polyvinyl alcohol to a freeze-thaw cross-linking step to eliminate the fluidity of the mixed solution; demoulding a product obtained from the freeze-thaw cross-linking step to obtain preformed microneedles; and subjecting the preformed microneedles to an irradiation cross-linking to obtain the cross-linked hydrogel microneedles.

Further, in the mixed solution, a mass concentration of polyvinyl alcohol is 1% to 30% and a mass concentration of sodium hyaluronate is 0.1% to 5%.

Further, the preparation method includes the following steps:

(1) preparing the mixed solution: adding polyvinyl alcohol into water and dissolving the polyvinyl alcohol solution in a water bath at 50° C. to 95° C. with stirring; adding sodium hyaluronate into water and dissolving to form the sodium hyaluronate solution with stirring; and mixing the sodium hyaluronate solution with the polyvinyl alcohol solution cooled to room temperature with stirring and defoaming under vacuum;

(2) freeze-thaw cross-linking: pouring the mixed solution obtained from step (1) into a mold, and performing freeze-thaw at 0° C. or below several times until obtaining a gel with no fluidity;

(3) demoulding the gel to obtain the preformed microneedles; and

(4) irradiation cross-linking: further cross-linking the preformed microneedles obtained from step (3) by irradiation to obtain stable hydrogel microneedles, and then performing dehydrating and drying to obtain a final product.

Further, a mode of the irradiation cross-linking is electron beam or gamma ray at an irradiation dose of 10 to 50 kGy.

Further, a drying method is one of blast air oven drying, oven drying, vacuum drying, and freeze drying.

According to a second aspect of the present disclosure, the following technical solutions are used in the present disclosure.

A cross-linked hydrogel microneedle for transdermal drug delivery is provided, which is prepared by the above preparation method.

According to the two-step cross-linking method of freeze-thaw and irradiation used by the present disclosure, the uncross-linked solution with fluidity is firstly freeze-thawed to obtain a preformed microneedle without fluidity, so that it can be released from a mold and the introduction of air bubbles during the transport process is avoided, and then the preformed hydrogel-like microneedle is further subjected to irradiation cross-linking to increase the stability of the microneedle, and the hydrogel microneedle is sterilized at the same time.

The present disclosure has the following beneficial effects:
(1) the hydrogel microneedles prepared by the two-step cross-linking method of freeze-thaw and irradiation according to the present disclosure are more stable and can withstand higher temperatures than those prepared by the freeze-thaw method alone, thus facilitating long-term transportation and preservation;
(2) in addition, by using the method in which cross-linking and shaping are firstly realized by freeze-thaw, air bubbles generated during transportation can be reduced and the yield can be increased when the irradiation method is subsequently performed; and
(3) through irradiation cross-linking, the hydrogel microneedles are sterilized while further shaping, so that the product is cleaner and safer for use without causing infection.

### DETAILED DESCRIPTION

The present disclosure is further described in detail below with reference to embodiments. The following embodiments are merely intended to describe the present disclosure rather than to limit the scope of the present disclosure. Non-essential improvements and adjustments made by a person skilled in the art under the core guiding ideology of the present disclosure still fall within the protection scope of the present disclosure.

### Example 1

### Preparation of Microneedles Through Irradiation Cross-Linking After Freeze-Thaw Preforming

(1) 10 g of polyvinyl alcohol 2499 was weighed, added into 80 ml of water for injection, placed in a water bath at 85° C. and stirred for 12 hours for dissolving, and then the solution was allowed to stand at room temperature and cooled;
(2) 0.05 g of sodium hyaluronate was weighed, added into 20 ml of water for injection and dissolved with stirring at room temperature to obtain a hyaluronic acid solution. The hyaluronic acid solution was then added to the polyvinyl alcohol solution, mixed with stirring at room temperature and defoamed under vacuum;
(3) the mixed solution was injected into a mold, the mold was placed into a refrigerator, frozen at −20° C. for 12 hours, then thawed at room temperature for 6 hours, and subjected to repeated freeze-thaw twice to eliminate the fluidity of the mixed solution so that the solution can be demolded, and then the preformed microneedles were demolded;
(4) the microneedles obtained from step (3) was subjected to irradiation cross-linking with Co-60 γ ray at an electron beam dose of 25 kGy; and
(5) the hydrogel microneedles obtained from step (4) were placed in a blast air oven and dried at 50° C. for 12 hours to obtain the final product.

### Example 2

### Preparation of Microneedles Through Irradiation Cross-Linking After Freeze-Thaw Curing

(1) 10 g of polyvinyl alcohol 2499 was weighed, added into 80 ml of water for injection, placed in a water bath at 85° C. and stirred for 12 hours for dissolving, and then the solution was allowed to stand at room temperature and cooled;
(2) 0.05 g of sodium hyaluronate was weighed, added into 20 ml of water for injection and dissolved with stirring at room temperature to obtain a hyaluronic acid solution. The hyaluronic acid solution was then added to the polyvinyl alcohol solution, mixed with stirring at room temperature and defoamed under vacuum;
(3) the mixed solution was injected into a mold, the mold was placed into a refrigerator, frozen at −20° C. for 12 hours, then thawed at room temperature for 6 hours, and cross-linked by repeated freeze-thaw 4 times, and then the fully cured hydrogel microneedles were obtained by demoulding;
(4) the hydrogel microneedles obtained from step (3) was subjected to irradiation cross-linking with Co-60 γ ray at an electron beam dose of 25 kGy; and
(5) the hydrogel microneedles obtained from step (4) were placed in a blast air oven and dried at 50° C. for 12 hours to obtain the final product.

### Comparative Example 1 Preparation of Microneedles Through Irradiation Cross-Linking Only

(1) 10 g of polyvinyl alcohol 2499 was weighed, added into 80 ml of water for injection, placed in a water bath at 85° C. and stirred for 12 hours for dissolving, and then the solution was allowed to stand at room temperature and cooled;
(2) 0.05 g of sodium hyaluronate was weighed, added into 20 ml of water for injection and dissolved with stirring at room temperature to obtain a hyaluronic acid solution. The hyaluronic acid solution was then added to the polyvinyl alcohol solution, mixed with stirring at room temperature and defoamed under vacuum;
(3) the mixed solution was injected into a mold and subjected to irradiation cross-linking with Co-60 γ ray at an electron beam dose of 25 kGy; and
(4) the hydrogel microneedles obtained from (3) were placed in a blast air oven and dried at 50° C. for 12 hours to obtain the final product.

### Example 3 Comparison of Number of Air Bubbles in the Microneedle

The numbers of air bubbles for the microneedles described in Example 1, Example 2, and Comparative Example 1 were compared. The above microneedles were cut into three 5 cm×5 cm squares, which were placed in a clarity tester to visually check the number of air bubbles under 1000 l×illumination. The results are shown in Table 1.

TABLE 1
Number of air bubbles

| | Number of air bubbles (pcs) | | | |
|---|---|---|---|---|
| Group | Sample 1 | Sample 2 | Sample 3 | Average |
| Example 1 | 6 | 4 | 4 | 4.67 |
| Example 2 | 2 | 2 | 3 | 2.33 |
| Comparative | 27 | 39 | 33 | 33 |
| Example 1 | | | | |

The above results show that the microneedles prepared by irradiation after freeze-thaw preforming are able to reduce the generation of air bubbles significantly compared to the microneedles prepared by direct irradiation.

The general description and specific examples used above are only used to help illustrate the present disclosure, and are intended to better explain the principles and practical applications of the present disclosure, rather than being used to limit the present disclosure. Modifications or improvements made by a person skilled in the art without departing from the spirit or essential features of the present disclosure should all fall within the scope of the present disclosure.

What is claimed is:

1. A method for preparing cross-linked hydrogel microneedles for transdermal drug delivery, comprising subjecting a mixed solution of sodium hyaluronate and polyvinyl alcohol to a freeze-thaw cross-linking step to eliminate the fluidity of the mixed solution; demoulding a product obtained from the freeze-thaw cross-linking step to obtain preformed microneedles; and subjecting the preformed microneedles to an irradiation cross-linking to obtain the cross-linked hydrogel microneedles.

2. The preparation method according to claim 1, wherein in the mixed solution, a mass concentration of polyvinyl alcohol is 1% to 30% and a mass concentration of sodium hyaluronate is 0.1% to 5%.

3. The preparation method according to claim 1, comprising the following steps:
(1) preparing the mixed solution: adding polyvinyl alcohol into water and dissolving the polyvinyl alcohol solution in a water bath at 50° C. to 95° C. with stirring; adding sodium hyaluronate into water and dissolving to form the sodium hyaluronate solution with stirring; and mixing the sodium hyaluronate solution with the polyvinyl alcohol solution cooled to room temperature with stirring and defoaming under vacuum;
(2) freeze-thaw cross-linking: pouring the mixed solution obtained from step (1) into a mold, and performing freeze-thaw at 0° C. or below several times until obtaining a gel with no fluidity;
(3) demoulding the gel to obtain the preformed microneedles; and
(4) irradiation cross-linking: further cross-linking the preformed microneedles obtained from step (3) by irradiation to obtain stable hydrogel microneedles, and then performing dehydrating and drying to obtain a final product.

4. The preparation method according to claim 1, wherein a mode of the irradiation cross-linking is electron beam or gamma ray at an irradiation dose of 10 to 50 kGy.

5. The preparation method according to claim 3, wherein a drying method is one of blast air oven drying, oven drying, vacuum drying, and freeze drying.

6. A cross-linked hydrogel microneedle for transdermal drug delivery, prepared by the preparation method according to claim 1.
